(19) United States
(12) Patent Application Publication
(10) Pub. No.: US 2013/0344559 A1
(43) Pub. Date: Dec. 26, 2013

(54) DEVICE AND METHOD FOR CONTROLLING NERVE GROWTH

(71) Applicants: Erik Daniel Engeberg, Cuyahoga Falls, OH (US); Rebecca Kuntz Willits, Akron, OH (US)

(72) Inventors: Erik Daniel Engeberg, Cuyahoga Falls, OH (US); Rebecca Kuntz Willits, Akron, OH (US)

(73) Assignee: THE UNIVERSITY OF AKRON, Akron, OH (US)

(21) Appl. No.: 13/868,511

(22) Filed: Apr. 23, 2013

Related U.S. Application Data
(60) Provisional application No. 61/636,939, filed on Apr. 23, 2012.

(51) Int. Cl. C12N 13/00

(52) U.S. Cl. CPC C12N 13/00
USPC 435/173.1; 435/289.1

(57) ABSTRACT

A nerve growth chamber includes a stimulation vessel defined by one or more sidewalls; at least one anode and at least one cathode positioned in the sidewall or sidewalls of the stimulation vessel; a conductive medium in the stimulation vessel; nerve tissue in the stimulation vessel; and a signal generator connecting the at least one anode and the at least one cathode and activated to generate a periodic AC signal at a desired frequency and amplitude, the periodic AC signal traveling through the conductive medium and affecting the growth of the nerve tissue. A method in accordance with the operation of the apparatus is also provided.

### CROSS-REFERENCE TO RELATED APPLICATIONS

This application claims priority from U.S. Provisional Application No. 61/636,939 filed on Apr. 23, 2012, the contents of which are incorporated herein by reference.

### FIELD OF THE INVENTION

The present invention generally relates to devices and methods for controlling nerve growth. More particularly, the present invention relates to devices exposing nerves to electrical stimulation to control their growth. The present invention also further relates to methods for controlling nerve growth through the application of electric stimulation at controlled amplitude and frequency.

### BACKGROUND OF THE INVENTION

The application of external stimulation in the form of electric fields has been known to impact neural growth and guidance in vivo as well as in vitro. Various studies have demonstrated that use of different external fields like direct current (DC), alternating current (AC) and pulsed electromagnetic fields (PEMF) influence neurite. In fact, application of electric stimulation for a short duration of 10 minutes has been shown to have significant growth in neurites. Also, studies have shown that the neurites have preferential and increased growth towards the cathode of an externally applied electric field. Electric stimulation can also have negative impact on nerve tissues. Experiments have been conducted where Xenopus laevis embryos were exposed to high amplitude electric fields that resulted in various abnormalities in the neural tube. Similar observations were made in the chick embryo neural.

Most of the electric stimulation studies have used DC to generate electric fields. Recent works on use of AC stimulation for enhancing neural properties have shown that AC stimulation is capable of producing electric fields of greater magnitudes over an increased distance than DC stimulation at the same current magnitude. The additional advantage of AC stimulation is decreased power consumption, which makes it more suitable for large scale therapeutic stimulation.

The results of the above mentioned studies indicate that application of electric stimulation in any form can have a large impact on the growth characteristics of nerve tissues. The present invention provides particular apparatus and methods to facilitate the growth of nerve tissue.

### SUMMARY OF THE INVENTION

A first embodiment of this invention provides an apparatus for controlling the growth of nerve tissue, the apparatus comprising:
a. a stimulation vessel defined by one or more sidewalls;
b. at least one anode and at least one cathode positioned in said one or more sidewalls;
c. a conductive medium in said vessel;
d. nerve tissue in said vessel; and
e. a signal generator connecting said at least one anode and said at least one cathode and activated to generate a periodic AC signal at a desired frequency and amplitude, said periodic AC signal traveling through said conductive medium and affecting the growth of said nerve tissue.

A second embodiment of this invention provides an apparatus as in the first embodiment, wherein the conductive medium is phosphate buffer saline.

A third embodiment of this invention provides an apparatus as in either the first or second embodiment, further comprising nerve growth factor in said stimulation vessel.

A fourth embodiment of this invention provides an apparatus as in any of the first through third embodiments, further comprising a plurality of stimulation vessels.

A fifth embodiment of this invention provides an apparatus as in any of the first through fourth embodiments, wherein said plurality of stimulation vessels are controlled by said signal generator.

A sixth embodiment of this invention provides an apparatus as in any of the first through fifth embodiments, further comprising a ceiling relative to said one or more sidewalls, and at least one of an anode or cathode in said ceiling and connected to said signal generator.

A seventh embodiment of this invention provides an apparatus as in any of the first through sixth embodiments, further comprising a floor relative to said one or more sidewalls, and at least one of an anode or cathode in said ceiling and connected to said signal generator.

An eighth embodiment of this invention provides an apparatus as in any of the first through seventh embodiments, wherein said conductive medium is hydrogel including a buffer solution providing conductivity.

A ninth embodiment of this invention provides an apparatus as in any of the first through eighth embodiments, wherein said conductive medium is biological tissue.

A tenth embodiment of this invention provides a method of guiding nerve growth, the method including the steps of:
a. subjecting nerve tissue to a periodic AC signal at a desired frequency and desired amplitude chosen to facilitate nerve growth as compared to the growth of the nerve without the application of such a periodic AC signal, the periodic AC signal travelling from a current source to a ground; and
b. guiding growth of the nerve tissue by positioning the nerve tissue between the current source and ground to effect desired growth in a direction toward the ground.

An eleventh embodiment of this invention provides a method as in the tenth embodiment, wherein said step of subjecting nerve tissue to a periodic AC signal includes the application of an AC signal across multiple pairings of current sources and grounds.

A twelfth embodiment of this invention provides a method as in either the tenth or eleventh embodiment, wherein the application of AC signals across multiple pairings is controlled by creating desired pairings of current sources and grounds and controlling which pairing is active at any given time.

A thirteenth embodiment of this invention provides a method as in any of the tenth through twelfth embodiments, further comprising the steps of: subjecting nerve tissue to a frustrating periodic AC signal at a desired frequency and desired amplitude chosen to frustrate nerve growth as compared to the growth of the nerve without the application of such a periodic AC signal, the periodic AC signal travelling from a current source to a ground.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a general schematic representation of an XY nerve growth chamber in accordance with the present invention.

FIG. 2 is a general schematic representation of the advancement of waveforms through a stimulation vessel wherein a single anode and multiple cathodes are positioned in a particular configuration.

FIG. 3 is a general schematic representation of the advancement of waveforms through a stimulation vessel wherein two anodes and two cathodes are positioned in a particular configuration.

FIG. 4 is a general schematic representation of the advancement of waveforms through a stimulation vessel wherein two anodes and two cathodes are positioned in a particular configuration.

FIG. 5 is a general drawing employed to describe the change in amplitude as a waveform advances from a single anode to a single cathode across conductive medium, and relates this change in intensity to the effects on growth of nerves positioned at positions A and C.

FIG. 6 is a general schematic representation of an XYZ nerve growth chamber in accordance with the present invention.

FIG. 7A is a schematic representation of the chamber as in FIG. 1, shown for describing the experimental procedures for measuring voltage at positions A and C for different frequencies.

FIGS. 7B-E provide graphs of the voltage a positions A and C and Rsens for all frequencies of stimulation, 20 Hz, 200 Hz, 1 MHz and 20 MHz. This experiment was done to ensure that the voltage and current passing through the DRG at different frequencies is the same, and that frequency is the only variable. A represents the circuit diagram of the experiment, where a resistor Rsens=98.3Ω was placed to near the cathode. The voltage across Rsens was used to calculate the current flow in the system using Ohm's law. (B-E) shows the voltage at positions A and C, and Rsens for all frequencies of stimulation. Results show 3.7% shift in the voltages for all the frequencies, which is negligible.

FIG. 8 is a bar graph depicting the average neurite growth and standard deviation for all frequencies of stimulation for positions A and C, as per FIG. 7A-E and the related experimental. The sample size was n=40 for each case. Statistical significance between the sham and stimulated samples is indicated by *(p<0.05). Results indicate that neurite length for 20 Hz, 200 Hz and 1 MHz (position A) is significantly different from sham and HF. Thus, LF stimulation promotes growth. Also, the length at position A was significantly different from position C for LF samples, indicating that high intensity fields aids in more growth of neurites.

FIG. 9 is a bar graph depicting the cell spreading ratio (CS) and standard deviation for all frequencies of stimulation for positions A and C. The sample size was n=40 for each case. Results indicate that CS ratio for stimulated samples is significantly different from the control samples. Thus, application of ES at any intensity or frequency helps in spreading of cells to greater distances, and the neurites follow them.

FIG. 10 is a bar graph depicting the average percentage of cell viability and standard deviation for all frequencies of stimulation for positions A and C. The sample size was n=40 for each case. Results indicate that the cell viability for stimulated samples is not significantly different from the control samples. Thus, application of ES at any intensity or frequency does not affect the cell viability.

FIGS. 11A-D are grey scale images of the DRG body and the surrounding neurites, with A being a zoomed out image thereof, B being a zoomed in and cropped image to reduce background, and C and D being left- and right-hand side images of FIG. 11B split into two equal parts. The number of white pixels in each half was counted and the ratio of cathode to anode (D) considered for analysis. If D is greater than 1, the neurites are denser towards the cathode.

FIG. 12 is a bar graph depicting the average density ratio (D) and standard deviation for all frequencies of stimulation for positions A and C. The sample size was n=40 for each case. Statistical significance between the sham and stimulated samples is indicated by *(p<0.05). Results indicate that density for 20 Hz, 200 Hz and 1 MHz (position A) is significantly different from sham and HF. Thus, LF stimulation leads to growth of denser neurites towards cathode. Also, D at position A was significantly different from position C for LF samples, indicating that high intensity fields aids in denser growth of neurites.

FIG. 13A is bar graph depicting the total number of neurite tips per quadrant for a sham and for frequencies of 20 Hz, 200 Hz, 1 MHz and 20 MHz. A illustrates the number of tips per quadrant for position A, while B represents position C. 400 neurites were counted for each condition. The plot representing sham is flat in both cases, indicating that neurites grow equally in all quadrants. The number of neurites is largest in quadrant 1 for 20 Hz and 200 Hz, indicating that more neurites grow to the side if the DRG facing cathode. The plots for 1 MHz and 20 MHz look similar to sham, depicting that HF does not promote directional growth of neurites.

### DETAILED DESCRIPTION OF ILLUSTRATIVE EMBODIMENTS

A first embodiment of an XY nerve growth chamber in accordance with the present invention is shown and designated by the numeral 10. The XY nerve growth chamber 10 is designated by “XY” to connote that the nerve growth is generally in a single planar direction. Though the nerve growth is generally radial and is of course three dimensional in the sense that it has volume, the direction of growth is concentrated generally in a single plane, designated here as an XY plane. More particularly, the chamber 10 includes a stimulation vessel 12 defined by opposed walls 14, 16, 18 and 20, and the direction of growth is concentrated in an XY plane from one or more anodes at one wall to one or more cathodes at other walls. Here, an anode 22 is shown positioned at the wall 14, and a cathode 24 is shown opposite the anode 22, at wall 16. More or less than four walls may be employed to create a stimulation vessel 12 with a desired polygonal or non-polygonal shape, with one or more anodes and cathodes positioned at one or more locations along the length of those walls. By way of example, optional anodes 26 and 28 could be employed at walls 18, 20, respectively.

In some embodiments, the walls of the vessel are formed from non-conductive materials. In other embodiments, the walls are formed from non-conductive materials selected from the group consisting of elastomers, such as silicone elastomers, polyurethane elastomers and polydimethylsiloxane.

In some embodiments, the anodes and cathodes are formed from materials selected from the group consisting of platinum, stainless steel, gold and silver.

A conductive medium 30 fills the vessel 12 and one or more nerves are placed in the conductive medium 30, necessarily between the anodes and cathodes, here represented by a single cathode 24 and multiple anodes 22, 26 and 28. In order to assist with the disclosure of this invention, two nerves are shown, a first nerve at “Position A”, labeled as nerve 32a, and a second nerve at “Position C”, labeled as nerve 32c. The nerve or nerves may be positioned between the anode(s) and cathode(s) in any suitable manner and at any desired location, though, as will be described herein, particular placements may be particularly desired. Thus, broadly, the nerve is provided in the vessel 12 and surrounded by the conductive medium 30. In more particular embodiments, the nerve or nerves are provided on or in a support 34 and positioned in the vessel 12 and surrounded by the conductive medium 30. In some embodiments, the support 34 is a coverslip and the nerve is adhered thereto. In other embodiments, the conductive medium 30 also serves as the support, such as by choosing a suitable hydrogel including a suitable buffer solution (such as phosphate buffer saline, PBS) as the conductive medium and depositing the nerve or nerves therein.

In some embodiments, nerve growth factor is also present in the stimulation to facilitate nerve growth.

At least one signal generator 36 is connected between all anodes and cathodes. The signal generator is chosen to apply a signal having any desired periodic alternating current (AC) waveform, such as sine, square, triangle and sawtooth, at desired frequencies and amplitudes. It may be desired to apply different signals across different pairings of anodes and cathodes and thus the signal generator preferably has outputs sufficient to supply discrete signals to any desired anode/cathode pairing.

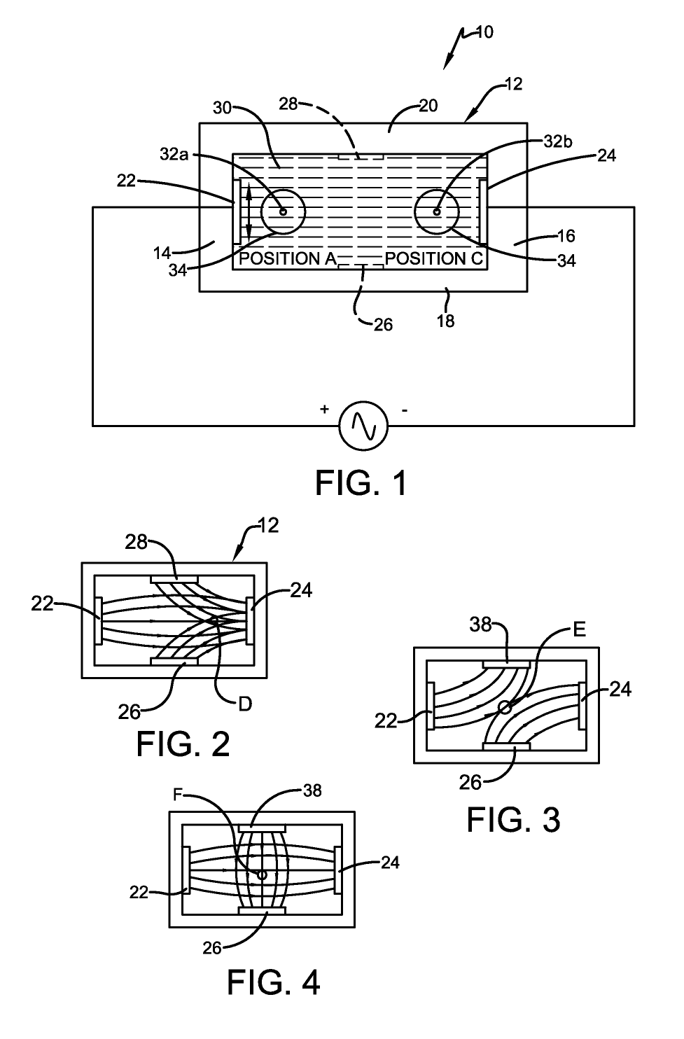

FIG. 2 provides a schematic for the dominant signal movement across the stimulation vessel 12 of FIG. 1. The optional anodes 26, 28 are employed in this case, and thus FIG. 2 shows the waves advance from all of the anodes 24, 26 and 28 to the single cathode 22.

FIG. 3 provides a schematic for the dominant signal movement across a stimulation vessel 12b wherein the anode 28 of vessel 12 is instead a cathode 38. The signal advancing from anode 22 tends to advance more toward the cathode 38, while the signal advancing from anode 26 tends to advance more toward the cathode 24, the tendency being fostered by the distance between these pairings.

In particular embodiments, the direction of signal movement is controlled by creating desired anode and cathode pairings and controlling which pairing is active at any given time. For example, though signal movement in FIG. 3 might be concentrated between the pairing of anode 22 and cathode 38 and the pairing of anode 26 and cathode 24 by the distance between those pairings, if both pairings are on at the same time, the signal will still at lest partially advance between anode 22 and cathode 24 and between anode 26 and cathode 38. Thus, to ensure the movement shown in FIG. 3, the pairing of anode 22 and cathode 38 can be turned on while the pairing of anode 26 and cathode 24 is turned off. Then the signal can be switched by turning the paring of anode 22 and cathode 38 off and the pairing of anode 26 and cathode 24 on. Pulsing (turning on and off) at very high frequencies can be used to good effect to create desired signal movement.

In FIG. 4, another example is shown where the pairings are anode 22 and cathode 24 and anode 26 and cathode 38. Signal movement can be controlled along the lines shown. Notably, with proper control of the signal generator, signal movement can be controlled to flip between the flows of FIG. 4 and those of FIG. 3.

For further control of signal movement, one or more of the cathodes or anodes can be made movable, as generally represented by the arrow at anode 22 in FIG. 1. Additionally, it should be appreciated that an array of such vessels can be provided as neighboring vessels to efficiently connect a single signal generator to multiple anodes and cathodes associated with each vessel. Thus, a plurality of vessels can be provided to grow a plurality of nerves, each vessel having one or more nerves therein for growth.

Notably, the signal movement affects the nerve growth, as the nerve, while naturally growing radially, will grow more quickly in the direction of signal movement—in the examples shown, toward the cathodes in the anode/cathode pairings.

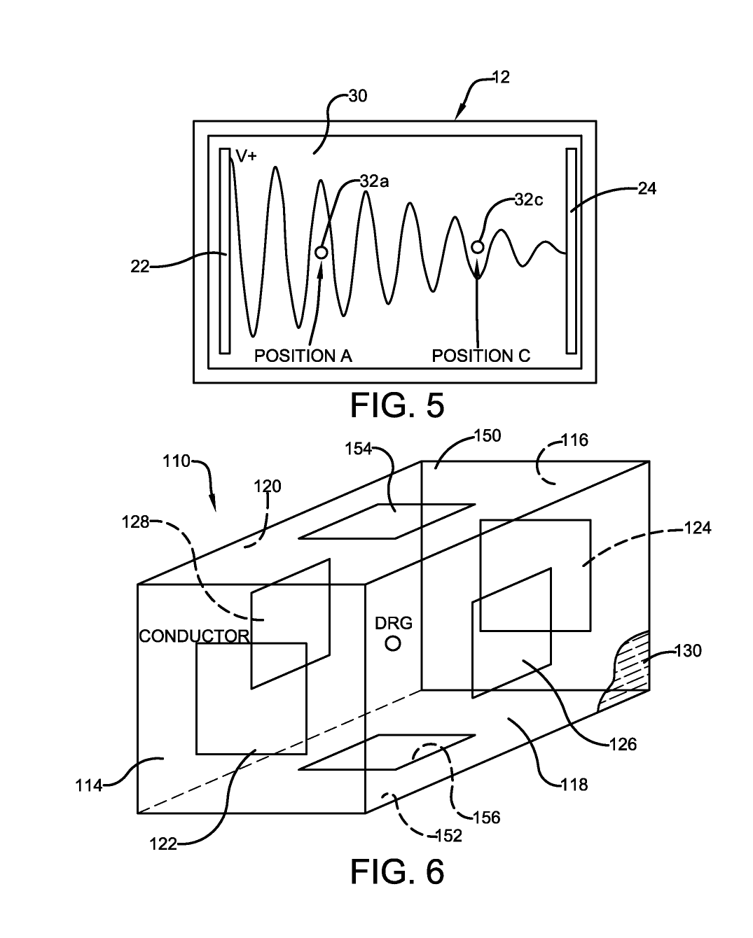

Referring now to FIG. 5, the stimulation vessel 12 is again shown, this time without the optional anodes 26 and 28 shown in FIG. 1. The advancement of a signal from the anode 22 to the cathode 24 is symbolically represented as well, and it can be seen that the amplitude (intensity) of the signal lessens as it travels through the conductive medium 30 to the cathode 24. Thus, it can be appreciated that the nerve 32a at Position A is exposed to an electric field of higher intensity/amplitude than the nerve 32c at Position C.

In experiments showing that a nerve positioned at Position A will grow faster than a nerve positioned at Position C, all other variables being controlled, it is deduced that the amplitude of electrical stimulation can affect nerve growth rate. Thus, in accordance with this invention, the positioning of the nerves in a stimulation vessel relative to one or more pairings of anodes and cathodes is employed to control nerve growth.

With the preceding explanation, the general concept of the invention should be well understood, along with the ability to adapt the concept to various desired growth patterns by the placement of anodes and cathodes along the wall or walls of a stimulation vessel. By way of example, a nerve placed at Position D of FIG. 2 would experience significant growth in the rightward direction, with the application of an appropriate signal. A nerve at Position E of FIG. 3 would tend to grow fastest toward an upward and rightward direction, with the application of an appropriate signal. A nerve at Position F in FIG. 4 will tend to grow more distinctively upward and rightward.

Turning now to FIG. 6, a second embodiment of this invention provides an XYZ nerve growth chamber designated by the numeral 110. In this embodiment, like parts to the XY nerve growth chamber 10 receives similar numerals, though increased by 100. The XYZ nerve growth chamber 110 is designated by “XYZ” to connote that the nerve growth is generally in multi-planar directions. Though the nerve growth is generally radial and is of course three dimensional, the direction of growth can be facilitated in various desired planes. More particularly, the chamber 110 includes a stimulation vessel 112 defined by opposed walls 114, 116, 118, 120, 150 and 152, and the direction of growth can be concentrated in various directions from one or more anodes at one wall to one or more cathodes at other walls. It would be appreciated that this embodiment basically adds a floor (wall 152) and ceiling (wall 150) to the general structure of the stimulation vessel 12 of the first embodiment. It will further be appreciated that this adds another dimensional component to the ability to position one or more anodes and cathodes. Here, an anode 122 is shown positioned at the wall 114, an anode 124 is shown positioned in wall 116, an anode 154 is shown positioned in wall 150, a cathode 126 is shown in wall 118, a cathode 128 is shown in wall 120 and a cathode 156 is shown in wall 152. More or less than six walls may be employed to create a stimulation vessel 112 with a desired shape, with one or more anodes and cathodes positioned at one or more locations along those walls. For further control of signal movement, one or more of the cathodes or anodes can be made movable, as already noted at anode 22 in FIG. 1. Not shown is one or more signal generators that interconnect the anodes and cathodes for desired signal movement, as described above with respect to the embodiment of FIG. 1.

The walls of the vessel can be formed from materials as already disclosed above for vessel 12. Similarly, the anodes and cathodes may be formed from the materials already disclosed above. Additionally, it should be appreciated that an array of such vessels can be provided as neighboring vessels to efficiently connect a single signal generator to multiple anodes and cathodes associated with each vessel. Thus, a plurality of vessels can be provided to grow a plurality of nerves, each vessel having one or more nerves therein for growth.

In this embodiment, the conductive medium 130 that fills the vessel 112 is preferably a hydrogel including a suitable buffer solution, the hydrogel providing support at a desired location therein and also permitting growth of the nerve therethrough. Thus, the nerve or nerves to be grown would be positioned in a hydrogel-filled vessel 112, as, for example, by depositing the nerves at a desired location with a syringe. Any electrically conductive medium could be used to support the nerves. In other embodiments, the conductive medium is biological tissue.

It has been found that the frequencies and amplitudes best suited for facilitating the growth of a given nerve may need to be experimentally determined for each nerve. That is, the best frequency and amplitude to facilitate growth may be different for different nerves. However, once experimentally determined, the desired frequencies and amplitudes can be applied across the nerve by appropriately positioning them relative to the anodes and cathodes of the stimulation vessels. Additionally, growth can be urged in a particular direction, as it has been found that the nerves grow faster in the direction of travel of the waveform (in this case from anode to cathode). This is shown in detail in the examples section presented herein.

With this understanding, in some embodiments, biologically possible frequencies on the order of 2 kHz or less are employed. In one aspect, the voltage amplitude of the applied stimulating signal used the present invention is also dependent upon the position of the nerve within the applied electric field created by the opposed anode and cathode. For example, a voltage amplitude of ˜2.1V at position A was found to achieve better nerve growth than a voltage amplitude of ˜0.9V at position C. It should also be appreciated that the resistance/impedance (R) of the electrically conductive medium supporting the nerves also impacts the growth rate of the treated nerves. For example, if the resistance/impedance (R) of the conductive medium is high, then a lower electrical current (I) would flow through the nerve being treated, since V (voltage)=I (current)*R (resistance/impedance). Thus, when considering a stimulating signal, both the amplitude of the voltage (V) and the amplitude of the current (I) are critical to achieving optimal nerve growth. As such, the amplitudes of voltage and current of the stimulating signal can be controlled using the chamber provided by the present invention. One manner for adjusting the change of resistance (R) is by increasing/decreasing the distance between the anode and cathode, or by utilizing a conductive medium used to support the nerves being treated that has an increased/decreased resistance (R). For example, if the distance between the anode and cathode of the chamber of the present invention used to treat the nerves is large, then the resistance (R) there between will be higher, resulting in a lower current flow (I).

Notably, the amplitude at the position of the nerve may be controlled by the positioning of the nerve, i.e., its spacing from the source of the wave to its ground (anode to cathode) or by controlling the intensity of the wave through the signal generator.

In light of the foregoing it will be appreciated that the present invention provides a method of guiding nerve growth, the method including the steps of subjecting nerve tissue to a periodic AC signal at a desired frequency and desired amplitude chosen to facilitate nerve growth as compared to the growth of the nerve without the application of periodic AC signal, the periodic AC signal travelling from a current source to a ground; and guiding growth of the nerve tissue by positioning the nerve tissue between the current source and ground to effect desired growth in a direction toward the ground.

Notably, just as certain frequencies can facilitate growth, other frequencies can be found to frustrate growth. By way of example, high frequencies of stimulation like 1 MHz, which are not biologically possible to generate, do not kill the cells, but rather just frustrate growth Therefore, the present method also entails, in some embodiments, applying a desired frequency that frustrates the growth of the nerve tissue at a non-desired region. With respect to the stimulation vessels disclosed herein, frequencies that frustrate the growth of the nerve tissue may be applied across certain anode/cathode pairs to frustrate growth in the region of the signals advancing between those pairs.

### EXAMPLES

### I. Materials and Methods

The fertilized eggs were obtained from Sunrise Farms, N.Y. F12K media, trypsin-EDTA and fetal bovine serum (FBS) were purchased from Sigma Aldrich and nerve growth factor (NGF, host species: mouse) was obtained from BD Biosciences. Sylgard 184 silicone elastomer kit (Cat #: SG1K) which contains the Sylgard 184 silicone elastomer base and Sylgard 184 silicone elastomer curing agent was obtained from ML Solar LLC. Sylgard 186 silicone elastomer kit (Cat #: DC4026144) which contains the Sylgard 186 silicone elastomer base and Sylgard 186 silicone elastomer curing agent was obtained from Dow Corning Silicones. Platinum foil (Cat #: 11509, 0.1 mm thick, 99.99% pure) was acquired from Alfa Aesar. Paraformaldehyde was obtained from Fisher (Cat #: T353-500), wetting agent Triton X-100 from Ricca Chemical Company (Cat #: 8698.5-16), sodium borohydride (Cat #: 102894) and bovine serum albumin (BSA, Cat #: 180577) were purchased from MP Biomedicals. Primary monoclonal antibodies neurofilament marker 3A10 (host species: mouse, isotype: IgG1), Schwann cell marker 1E8 (host species: mouse, isotype: IgG1) and fibronectin marker B3/D6 (host species: mouse, isotype: IgG2a) were obtained from Developmental Studies Hybridoma Bank, University of Iowa. Secondary antibodies Alexa Flour 488 goat anti-mouse IgG1 (Cat #: A21121) and Alexa Flour 546 goat anti-mouse IgG2a (Cat #: A21133) were purchased from Molecular Probes. DNA stain Hoechst 33342 (Cat #: H1399) was purchased from Life Technologies. Live/dead stains Calcein AM (Cat #: ALX-610-026-M001) was obtained from Enzo Life Sciences and Ethidium Homodimer (Cat #: E1169) from Life Technologies.

A. Tissue Culture

The DRG were harvested from E9 chick embryos, and placed on circular glass coverslips (13 mm) with minimal fluid (˜100 n1) and incubated at 37° C., 5% CO2 for 4 hr to encourage attachment. The coverslips were acid etched prior to use by washing them with soapy water and blow drying them with air. The coverslips were immersed in 9:1 H2SO4:H2O2 for 20 mins, washed in deionized water and submerged in absolute ethanol. Thereafter, the coverslips were blow dried completely before use. Also, the bottom side of each coverslip was marked using a diamond tip pen, which indicated the field alignment of the coverslip towards the negative electrode. The coverslips were treated with collagen (3.156 mg/mL rat type I in 0.02M acetic acid) overnight at 150 μg/mL concentration in phosphate buffered saline (PBS), at 4° C. This aided in the attachment of the DRG to the surface of the coverslip. The DRG containing coverslips were cultured for 24 hr in growth medium containing Ham's F-12K and 20% FBS, with NGF at 25 ng/mL concentration, at 37° C., 5% CO2. A coverslip was placed in one plate of a 24 well plate and covered with 0.5 mL of NGF-supplemented media. After 24 hr, the pre-stimulation images were taken for all the cultured DRG.

B. Electric Chamber Fabrication

The rectangular silicone chamber was fabricated to stimulate the DRG using Sylgard elastomers. The Sylgard 184 and 186 bases were mixed with their respective curing agents at 9:1 ratio in a flat bottomed plastic container to obtain 5 mL volume for each elastomer. The elastomers 184 and 186 were combined together at 1:1 ratio to obtain 10 mL volume of the mixture and placed in a vacuum desiccator for 10 mins to remove the air bubbles in the elastomer mixture. Thereafter, the mixture was spread out evenly with 1 cm thickness on a mold with a 7×3 cm void in the center and allowed to gel at 37° C. overnight. The gel was removed slowly from the mold and copper wires were inserted from the left and right parts of the chamber. Platinum sheets were cut into 3×1 cm rectangular shapes and soldered to the copper wire on either side of the chamber. The chamber was then washed with DI water and soaked in 70% ethanol for 15 mins while being gently shaken. The chambers were rinsed with DI water and soaked in acetone for 15 mins on the shaker. After that, the chambers were rinsed in ultra-pure DI water and dried in the oven at 60° C. for 1 hr. While warm, the chambers were placed firmly on top of acid-etched glass slides. The glass slides with the chamber on top were wrapped in Kimwipe, placed in a sterilization pouch and autoclaved. The cleaning procedure for the chambers was followed before each experiment.

C. Electric Stimulation

The cultured DRG were stimulated at different frequencies in the electric chamber. After autoclaving the chambers, two points were marked on the bottom of the glass slide, at a distance of 0.5 cm from each electrode using a marker. These points represented the positions of the coverslips at the anode and cathode—designated as position A and position C, respectively. This was done to maintain a constant distance of the DRG from the electrode to expose the tissue to consistent electric fields.

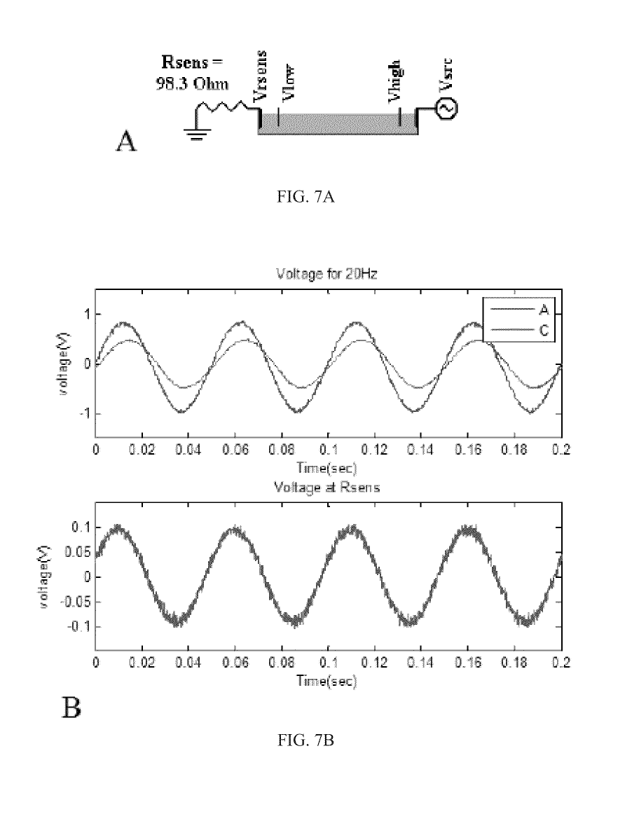

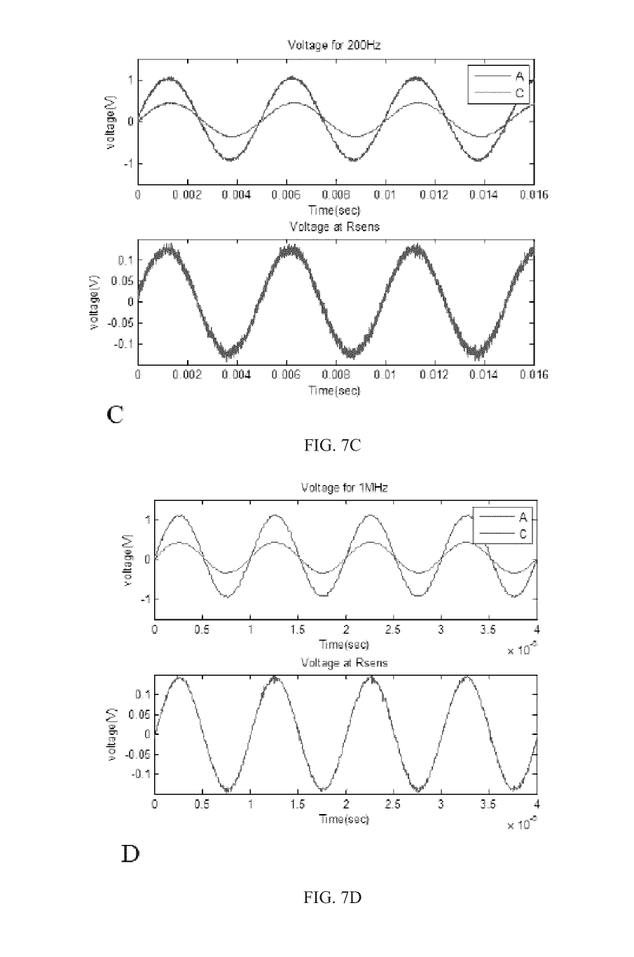

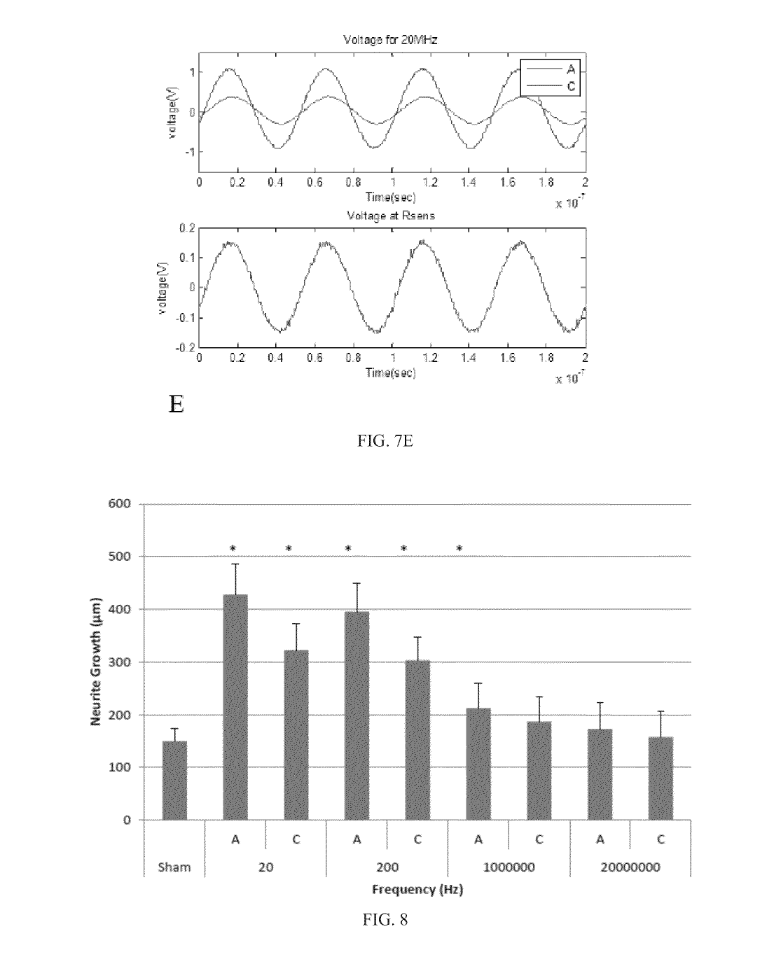

The chamber was filled with 1.5 mL of PBS and two DRG containing coverslips were placed on positions A and C, similarly to as shown in FIG. 1 (and as represented in FIG. 7A). A sinusoidal input was passed between the electrodes at 4 different frequencies—20 Hz, 200 Hz, 1 MHz and 100 MHz—at constant amplitude of 2.5 Vp-p. All stimulations were done for 1 hr. The voltage at positions A and C was measured by connecting two wires at A and C with the oscilloscope. To determine the current in the system for each frequency, a low resistor of value 98.3Ω was connected with the cathode. The voltages were measured at four points for all frequencies, as shown in FIGS. 7A-E, wherein, as per FIG. 7A:

Vsrc=Input Voltage

Vhigh=Voltage at position A

Vlow=Voltage at position C

Vrsens=Voltage at the sensor (resistor)

The current in the system, Irsens for each frequency was calculated by:

Iᵣₛₑₙₛ=Vᵣₛₑₙₛ/Rₛₑₙₛ

Where Rsens=98.3Ω. The results are listed in Table 1.

TABLE 1
Voltage and current measurements in the ES chamber
for different frequencies at positions A and C

| Freq (Hz) | position A (V) | position C (V) | Vrsens(V) | I rsens(A) |
|---|---|---|---|---|
| 20 | 2.04 | 0.92 | 0.2 | 0.002035 |
| 200 | 2.08 | 0.9 | 0.268 | 0.002726 |
| 1000000 | 2.10 | 0.88 | 0.288 | 0.00293 |
| 20000000 | 2.12 | 0.86 | 0.312 | 0.003174 |

Sham exposed controls were examined for each frequency where the DRG containing coverslips were placed in the chamber for 1 hr, in PBS with no electric stimulation. The controls were tested to ensure that the changes in the tissue were due to the direct effect of stimulation and not due to any byproducts generated by stimulation of the media. Following stimulation, the DRG were cultured for 24 hr in NGF-supplemented growth media (nerve growth factor-supplemented).

D. Fixation and Staining

Post-stimulated DRG were fixed using 4% paraformaldehyde, which was freshly prepared by dissolving 0.4 g of paraformaldehyde powder and 0.1M NaOH in 0.7 mL of tissue culture water at 60° C. The solution was cooled at room temperature after adding 1 mL of 10×PBS and the pH was brought to 7.4 by adding 0.1M HCl. Tissue culture water was added to bring the volume to 10 mL. The DRG were placed in paraformaldehyde for 20 mins and were rinsed with PBS two times to remove any residual aldehyde. The tissues were permeabilized with 0.5% Triton X-100. Freshly prepared sodium borohydride was added to quench the aldehyde. Finally, 3% BSA was used as a blocking agent, to restrict non-specific binding of the antibodies.

Antibody Staining: After fixation, a neurofilament marker monoclonal primary antibody 3A10 was added to the DRG, at a concentration of 1:200 in 1×PBS. The DRG were incubated overnight at 4° C. After that, the secondary antibody—Alexa Fluor 488 was added to the DRG, diluted at 1:200 in 1×PBS, and incubated for 3 hr at 37° C. To label the cell, DNA stain H33342 was added along with the secondary antibody at 1:1000 dilution.

Viability test: The viability tests were done using 4 mM Calcein AM and 2 mM Ethidium Homodimer. The concentrations of the solutions were taken as 10 μM and 4 μM for Calcein AM and Ethidium Homodimer, respectively. The solutions were diluted with 1×PBS from the stock concentration to their respective working concentrations. The solutions were then mixed with DNA stain H33342. About 200 μl of the mixed solution was placed over the coverslips. The coverslips were incubated at room temperature for 45 mins, and then imaged.

E. Image Analysis

The DRG were imaged using Carl Zeiss inverted optical microscope. Phase images were taken for pre-stimulated samples using 5× magnification. Fluorescent mosaic images (4×4) were taken for all the post-stimulated DRG samples to cover the entire neurite growth and cells. The images were analyzed for length of the longest neurite, distance of the furthest cell, cell viability and density of neurite growth with respect to the electric field. Axiovision 4.8 image processing software was used for all image analysis.

F. Neurite Growth

The growth of the neurite was determined by obtaining the difference between the length of longest neurite in pre-stimulated and post-stimulated images. The length of the longest neurite was measured by drawing two concentric circles on the DRG—the inner circle representing the radius of the DRG body and the outer circle representing the tip of the longest neurite. The inner and outer radii for pre-stimulated images were taken as i₁ and O₁, and for post-stimulated images, i₂ and O₂. The growth of the neurite was calculated using the following formula:

Growth=(O₂−i₂)−(O₁−i₁)

The average growth for the corresponding number of samples for each position and control was calculated, and the results were plotted, as shown in FIG. 8.

G. Cell Spreading

The effect of electrical stimulation on movement of the cells from the DRG body was determined in this analysis. This analysis gives quantification for the spreading of the cells in stimulated samples and compares it with the control samples. It determines how much distance the cells have moved in electrically stimulated samples, with respect to the length of the longest neurite. The distance of the furthest cell was measured in the same procedure as the length of the longest neurite—an inner circle was drawn representing the radius of the DRG body (i₃) and an outer circle representing the distance of the cell furthest away from the DRG body (O₃).

L_{C}=(O₃−i₃)

where L_{C} is the distance of the furthest cell from the DRG body.

Since the pre-stimulated images are not stained for cell bodies, only post-stimulated images were considered for this analysis. In order to compare the distance of the furthest cell from the length of the longest neurite, the ratio of the distance of the cell migration to the length of the longest neurite was taken. It must be noted that for the ratio, the length of the longest neurite is considered, not the growth. The length of the longest neurite in the post-stimulated image was determined by:

L_{N}=(O₂−i₂)

where i₂ is the radius of the DRG body, and O₂ is the radius of the circle representing the length of the longest neurite.

The cell spreading ratio was determined by using the following formula:

CS=L_{C}/L_{N}

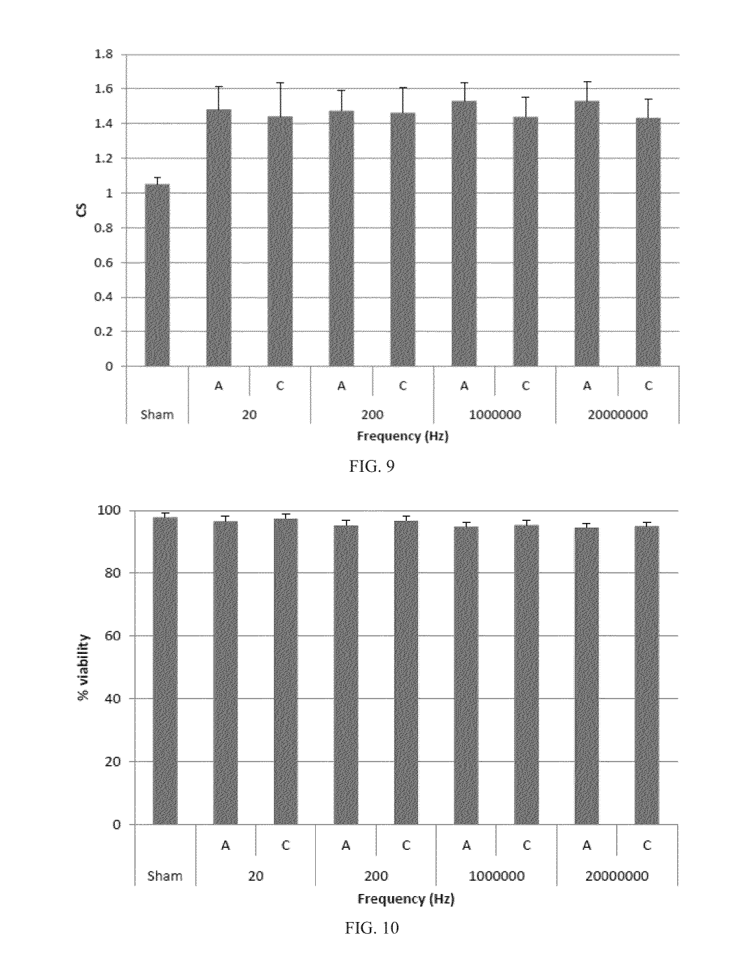

It must be noted that if the value of CS is greater than 1, the distance of the cell movement is more than that of the longest neurite; if it is less than 1, the length of the neurite is more than the furthest cell; and if it is equal to 1, the distance of the furthest cell is same as the length of the longest neurite. The ratio was taken separately for positions A and C, as well as for sham control. The average value of CS for all the samples tested was plotted with respect to frequency for positions A and C, as shown in FIG. 9.

H. Cell Viability

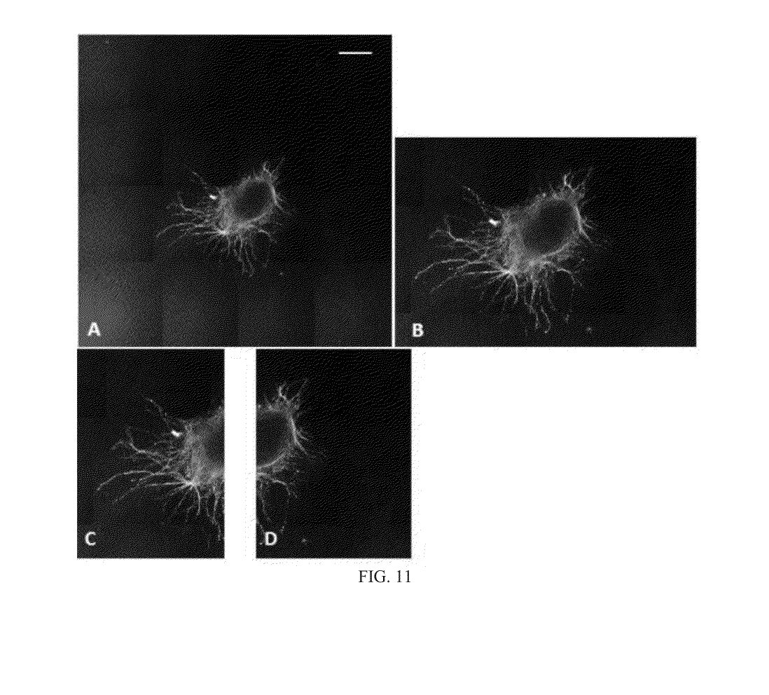

The quantification of cell viability after exposure to stimulation was done by determining the % cell viability for stimulated samples of all frequencies, at positions A and C, and comparing it with the controls. The average±standard deviation % viability was calculated and plotted, as shown in FIG. 10.

I. Neurite Density

The density of neurite growth with respect to the direction of current flow was determined MATLAB and ImageJ were used for the quantification of neurite density for all the frequencies of stimulation. To measure density, an image 3700×3700 pixel units was loaded in ImageJ. The DRG body was traced using the ellipse option and removed. The new image now contained only the neurites. The image is converted into grey scale image for easy analysis. The original image is shown in FIG. 11. The image is zoomed and the area of the DRG was cropped so that all the neurites are enclosed in the new image, as shown in FIG. 11B. The x- and y-coordinates of the center pixel was determined and the image was split into two equal parts along the y axis, as shown in FIG. 11 (C-D). One half is the anode facing side of the DRG while the other is the cathode facing side. Since the neurites have stained green, the intensity of white pixels in each half was measured using MATLAB. A variable ‘count’ was defined to keep track of the number of white pixels. Thus, the value of ‘count’ gave the number of white pixels in each half of the image. The density ratio, D, of neurites was calculated by:

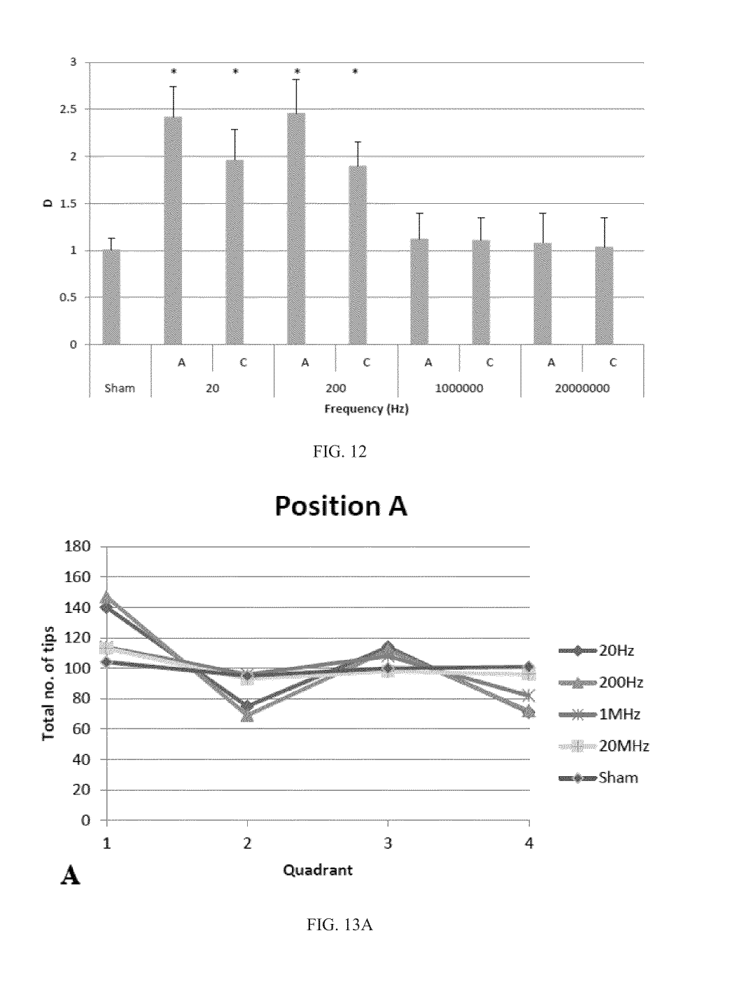

D=count C/count A

Where count C is the number of white pixels in the image half facing cathode, count A is the number. of white pixels in the image half facing anode. If D>1, the density of neurites is towards the cathode, if D=1, there is equal density distribution of neurites both sides and if D<1, the density of neurites is more towards the anode. The average of D for all frequencies and positions was calculated and statistical tests were performed, as shown in FIG. 12.

J. Neurite Directionality

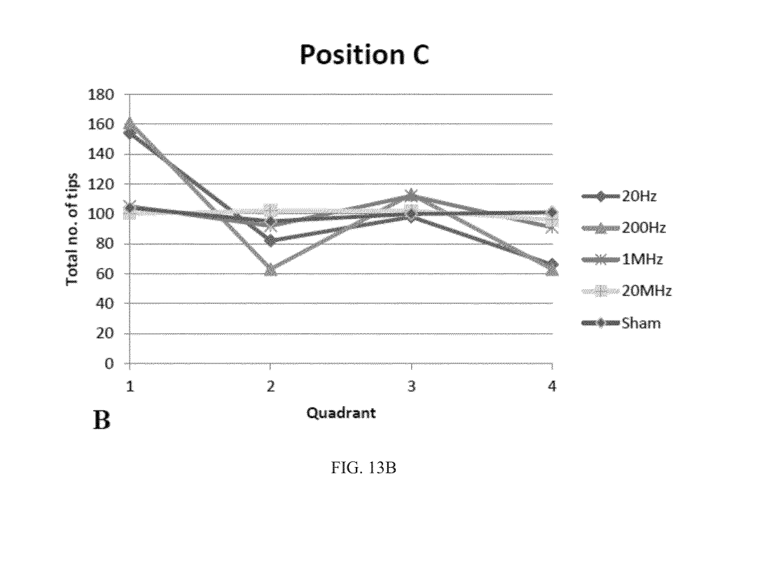

The orientation of the neurite growth towards anode or cathode was quantified using polar plots. For an image, 10 longest neurites were considered for directionality analysis, 5 for each half of the image. The neurites were traced using ImageJ to determine the length, which was represented as r. The angle of the neurite tip from the center of the DRG body was measured, and denoted as θ. The polar coordinates of the tip of each neurite (r, θ) were plotted as points in polar plots with 10° intervals using MATLAB. The polar plot was divided into 4 quadrants. Quadrant 1 faced the cathode which was treated as positioned at 0° with quadrant 1 spanning approximately from 45° to 315°. Quadrant 3 faced the anode which was treated as positioned at 180° with quadrant 3 spanning approximately from 135° to 225°. Quadrant 2 spanned approximately from 45° to 135°. Quadrant 4 spanned approximately from 225° to 315°. In creating the polar plots, each point plotted represented a group of 20 neurite tips per 30° i.e. for every 30°, 20 tips were counted and represented as one point with a standard deviation of ±10°. The number of points per quadrant (Ntips) was measured and added up to see which quadrant had the highest number of neurites, for all the frequencies and positions. The total number of neurite tips per quadrant was plotted for each frequency for positions A and C, as shown in FIG. 13A and FIG. 13B.

K. Statistical Analysis

Statistical analysis was done on the average values of neurite growth, cell spreading, neurite density, directionality and cell viability for each position and frequency of stimulation using SAS. A non-parametric test called the Kruskal-Wallis test was employed to obtain significance between the data, where p<0.05 was considered significant.

### II. Results

A. Neurite Growth

The graphical representation of average neurite growth for about 40 samples per condition, 24 hr after stimulation with respect to the stimulation frequency and position is showed in FIG. 8. The average growth for 20 Hz and 200 Hz was 427±58.07 mm and 395±54.78 mm and it decreased with an increase of the input frequency of stimulation. Also, the neurite growth was comparatively greater for DRG at position A than position C, at low frequencies. Statistical tests showed significant difference between the neurite growth at positions A and C for 20 Hz and 200 Hz (p<0.05). However, no significant difference was found between the neurite growth at positions A and C for 1 MHz and 10 MHz (p>0.05). Since DRG at position A is exposed to higher EF than position C, these results indicate that neurites grow longer at regions exposed to higher EF. However, the effect of EF was prominent only at low frequencies. The neurite length was also significantly different for stimulated samples from the control (149±23.97 μm) for 20 Hz, 200 Hz and position A at 1 MHz, but not for positions C for 1 MHz and A, C for 20 MHz (p>0.05). Thus, AC stimulation of nerve tissue at frequency less than 1 MHz increases neurite growth. The growth of the neurite at 1 MHz and 20 MHz was almost similar to the control samples. The results are tabulated in Table 2.

TABLE 2
Average ± standard deviation neurite
growth for all frequencies and positions.

| Freq (Hz) | Sham | position A (μm) | position C (μm) |
|---|---|---|---|
| | 149.65 ± 23.97 | — | — |
| 20 | — | 427.61 ± 58.07* | 322.08 ± 50.25* |
| 200 | — | 395.31 ± 54.78* | 302.94 ± 44.64* |
| 1000000 | — | 212.51 ± 47.13* | 186 ± 47.39 |
| 20000000 | — | 172.54 ± 51.33 | 157.54 ± 48.65 |

(*represents the values that are significantly different from sham.)

B. Cell Spreading

The cell spreading ratio (CS ratio) was calculated for 40 samples per condition, and the average CS ratio was plotted with respect to frequency and position, as shown in FIG. 9. The CS ratio for control sample was calculated as 1.05±0.03, which is very close to 1, indicating that the distance of the furthest cell was same as the neurite length. Statistical analysis showed no significant difference in the CS ratio between positions A and C for all the frequencies of stimulation (p>0.05). However, statistical significance was observed between CS ratio of stimulated samples for all frequencies with the control samples, with average CS ratio of 1.48±0.04 (p<0.05). Thus in culture, the cells of the DRG spread away from the DRG body. The distance of the furthest cell from the center of the DRG body was more than the longest neurite in stimulated samples. This indicates that AC stimulation causes the cells in the tissue to spread at a greater distance than no stimulation, regardless of the stimulation frequency and the magnitude of the EF. The growth of the neurites follows the path of the cells, which may indicate the possibility of the cell movement acting as guidance cues for the neurites.

C. Cell Viability

FIG. 10 depicts the % viability of cells after stimulation at positions A and C. The plot shows that 96±1.5% cells were viable after 1 hr AC stimulation at 20 Hz and 200 Hz. The % cell viability was not significantly different for lower frequencies than at 1 MHz and 20 MHz, which were 95±1.35% and 94.45±1.4%, respectively (p>0.05). The results show that high frequency AC stimulation of nerve tissue does not impact the cell viability significantly as compared to low frequencies. The results are tabulated in Table 3.

TABLE 3
Average ± standard deviation % viability
for all frequencies and positions.

| Freq (Hz) | Sham | position A (μm) | position C (μm) |
|---|---|---|---|
| | 97.8 ± 1.4 | — | — |
| 20 | — | 96.9 ± 1.6 | 97.4 ± 1.3 |
| 200 | — | 95.2 ± 1.5 | 96.7 ± 1.4 |
| 1000000 | — | 94.8 ± 1.3 | 95.3 ± 1.4 |
| 20000000 | — | 94.5 ± 1.5 | 94.9 ± 1.3 |

D. Neurite Density

The density of the neurites with respect to the position of the electrode was quantified for each frequency using density ratio, D. D was calculated for all the 40 experiments for each frequency and sham, and the average D±standard deviation for each frequency was plotted. The value of D was found to be 1.009±0.27 for control, indicating that the density of neurite growth towards anode and cathode from the center of the DRG body is almost same. However, for low frequencies, D was around 1.9±0.32 for position C and 2.4±0.37 for position A, which was significantly different from control and HF (p<0.05). These results indicate that neurites grow more densely towards cathode than anode, and that the density is greater near anode. The reason for this may be the movement of charge in the electrolyte is from anode to cathode which drives the neurites towards cathode. Also, the density of neurites at position A is greater due to more EF intensity at A than C, which favors neurite growth. As the frequency increases to MHz range, the ratio becomes 1.1±0.29 for position A and 1.12±0.27 for position C. The density results are similar to those obtained in length of neurite outgrowth, where an increase of frequency decreases growth and the characteristics of the tissue become similar to control (p>0.05). Thus, HF simulation reduces the length as well as density of neurite growth. The plots for D vs frequency are tabulated in Table 4.

TABLE 4
Average ± standard deviation density
ratio (D) for all frequencies and positions.

| Freq (Hz) | Sham | position A (μm) | position C (μm) |
|---|---|---|---|
| | 1.01 ± 0.12 | — | — |
| 20 | — | 2.41 ± 0.32* | 1.96 ± 0.32* |
| 200 | — | 2.45 ± 0.36* | 1.89 ± 0.25* |
| 1000000 | — | 1.12 ± 0.27 | 1.11 ± 0.24 |
| 20000000 | — | 1.08 ± 0.32 | 1.04 ± 0.31 |

(*represents the values that are significantly different from sham.)

E. Neurite Directionality

The quantification of orientation of neurites with regard to the direction of EF was done using polar plots, where the distance of the tip of the neurite from the DRG body and the angle of the tip were considered. Ntips is same for sham, as the line representing sham is almost flat. The results for HF are almost equal to sham control, with about 103 neurites in quadrant 1 for C and 113 for A. Thus for HF, the number of neurites in each quadrant is not significantly different from each other as well as the sham (p>0.05). On the other hand, Ntips for 20 Hz and 200 Hz stimulation for position A in quadrant 1 are 140 and 147, respectively, which is significantly greater than the Ntips for all the other quadrants (p<0.05). The Ntips for position C for low frequency stimulation is about 157±4, which is significantly different from all other quadrants, as well as HF stimulation (p<0.05). Thus, the number of neurites growing in quadrant 1 is significantly more than all the other quadrants and since quadrant 1 faces the cathode, it indicates that neurites have directionality towards the cathode i.e. the growth follows the direction of EF. Also, directionality is observed in LF and as the frequency increases, the neurites grow symmetrically around the DRG body, similar to sham. The results are tabulated in Table 5.

TABLE 5
Total number of neurites in 4 quadrants.

| | | Quad 1 | Quad 2 | Quad 3 | Quad 4 |
|---|---|---|---|---|---|
| Sham | | 104 | 95 | 100 | 101 |
| 20 | A | 140* | 75 | 114 | 71 |
| | C | 154* | 82 | 98 | 66 |
| 200 | A | 147* | 69 | 112 | 72 |
| | C | 161* | 63 | 113 | 63 |
| 1000000 | A | 114 | 96 | 108 | 82 |
| | C | 105 | 92 | 112 | 91 |
| 20000000 | A | 113 | 93 | 98 | 96 |
| | C | 100 | 102 | 102 | 96 |

(*represents the values that are significantly different from sham.)

### III. Discussion/Conclusions

The effect of low and high frequency stimulation on dielectric and biological properties of chick DRG was quantified in this study. DRG was modeled using Maxwell-Wagner and Cole-Cole models and their corresponding conductivity with respect to the input frequency was calculated. These models had two branches: capacitive and resistive. The resistive branch represented the ECF while the capacitive branch represented the cell membrane. During DC stimulation, membrane capacitance tissue blocks DC current and all the current flows through the resistive element, which is the ECF. At low frequencies, the current flow in the tissue depends on the charging and discharging properties of the capacitive component. At higher frequencies, the capacitive component of the tissue is diminished, which decreases the overall impedance of the tissue thereby affecting the voltage and current passing through the cell.

Results of the present study showed that ES impacts length, cell movement, density of growth and orientation of growth, when compared to control samples. The cell spread more away from the DRG body at greater distances than the neurites on the application of AC stimulation, regardless of the frequency of stimulation and intensity of EF. It has been suggested by Marin that movement of cells aids in neurite growth by steering the growth cone, application of EF leads to spreading of cells to greater distances in less time. This may aid in growth of the neurites to longer lengths. At LF, the cells spread out to great distances and the neurites seem to follow them resulting in increased growth. However, at HF, even though the cells spread out more than the neurites, the inability of the neurite membrane to adjust to the rapid input cycles may have a more prominent effect on the length, which resulted in stunted growth.

Neurite growth and density was enhanced at LF stimulation, with longer and denser neurites at position A than position C. At HF, the growth and symmetry was almost same as control, and less than LF. The value of average neurite growth at LF was found to be around 411±22.84 μm, which was similar to the results obtained by studies on DC and LF stimulation. Research done by Graves et al has demonstrated that the length of neurite growth is similar up to 1 KHz input frequency. However, the growth at frequencies greater than 1 KHz was not reported in that study.

Since the impact of DC and LF stimulation is similar, it may be because the path of the current during DC and LF stimulation is the same. According to Grimnes et al, at DC and LF fields, the current passes through the ECF as the membrane capacitance blocks the current to pass into the tissue. Also, the tissue allows less current to pass through it as the conductivity of the tissue at LF is less. These results indicate that LF stimulation encourages positive neurite response in terms of growth than HF. Also, since more growth and denser neurites were observed at position A than position C, the intensity of the EF applied also plays an important role in the tissue response to EF. Since the conductivity of the tissue is low at LF and increases significantly as the frequency increases, the amount of current flow inside the tissue also impacts growth. Greater EF intensity and low current promotes growth of neurites. Several hypotheses have been suggested to describe the reason of the increase in neurite growth. Koppes et al. proposed that EF increases the production of nerve growth factors (NGF) in SCs which promotes neurite growth. Neurite extension can also be influenced by a variety of factors such as cellular participation, substrate, soluble factors, external forces, and topography. According to Lidan et al, an upregulation of Ca2+ mediated brain derived neurotrophic factor due to LF stimulation enhances growth and density of neurites.

The flow of charge inside the ES chamber is from anode to cathode. The results from the present study shows that neurites exhibit preferential cathodal growth under the influence of LF AC fields, which is similar to DC fields. The number of neurites in quadrant 1 was not significantly different from the other quadrants in case of HF stimulation. Research is still ongoing to establish the cellular processes involved in explaining the cathodal preferences of growth. One of the hypothesis was formulated by Patel et al. which suggested the cathodal accumulation of growth-controlling surface glycoproteins by EF is the underlying mechanism of the field-induced orientation of neurite growth towards the cathode.

Thus, LF impacts the length, density and cathodal directionality of neurites However, at HF the neurites do not show any directionality, indicating that at higher conductivities, neurites do not respond fast enough to the changes in the EF. During LF stimulation, each wave of the AC signal passes through the tissue at a slow rate giving the cells ample time to respond to the changes in the surrounding potential. Under the influence of ES, the ion channels open and close at a faster rate than normal and this leads to greater intake of ions inside the membrane. As the input frequency increases, the signal cycles fall on the cells at a much faster rate, which does not give the cells enough time to respond to the surrounding potential changes. The ion channels are unable to open and close at a rate corresponding to the input frequency. Moreover, an increase of conductivity at HF would lead to intake of higher currents through the cell membrane, thus changing the potential to an even higher degree. The inability of the nerve tissue to respond to the quickly changing environment may have an impact on the growth, density and directionality of the neurites.

From the perspective of using AC stimulation in treatment of nerve injury, even though LF stimulation shows similar cellular response to DC stimulation, AC stimulation has additional benefits of low power consumption and greater penetration. Graves et al demonstrated that greater EFs are established by AC stimulation at equal current magnitudes to DC stimulation, which indicates that similar fields can be achieved with less AC stimulation than DC thereby reducing the power. According to this study, the decrease in power consumption would decrease the size of the implantable device due to smaller battery requirements and allow for longer battery life and greater treatment periods. Because higher frequencies of stimulation tend to reduce the impedance of most types of biological tissue, lower power levels can be used to drive more current through the tissue with high frequencies of stimulation.

In the present study, the effect of AC stimulation on chick DRG was observed, at LF and HF in a non-uniform EF. Results suggest that LF stimulation enhances neurite growth and density at higher EF intensity. Also, AC stimulation causes the cells to move away from the DRG body and the neurites slowly follow the cells, which indicate that the EF leads to faster movement of cells that can act as a guidance factor for the neurites. LF stimulation of DRG also aids the neurites to grow preferentially towards the cathode, following the charge flow. The biological characteristics of the DRG at HF are almost the same as control conditions. The hypothesis of this study is that these characteristics are influenced by the changes in conductivity of the DRG at HF. Due to the inability of the cells to respond quickly to the increased rate of cycles at HF and greater currents inside the tissue, the cellular response of the tissue at HF is diminished than LF. Thus, high intensity fields at LF enhance biological characteristics of nerve tissues better than low intensity fields and HF stimulation.

What is claimed is:

1. An apparatus for controlling the growth of nerve tissue, the apparatus comprising:
a. a stimulation vessel defined by one or more sidewalls;
b. at least one anode and at least one cathode positioned in said one or more sidewalls;
c. a conductive medium in said vessel;
d. nerve tissue in said vessel; and
e. a signal generator connecting said at least one anode and said at least one cathode and activated to generate a periodic AC signal at a desired frequency and amplitude, said periodic AC signal traveling through said conductive medium and affecting the growth of said nerve tissue.

2. The apparatus of claim 1, wherein the conductive medium is phosphate buffer saline.

3. The apparatus of claim 1, further comprising nerve growth factor in said stimulation vessel.

4. The apparatus of claim 1, further comprising a plurality of stimulation vessels.

5. The apparatus of claim 4, wherein said plurality of stimulation vessels are controlled by said signal generator.

6. The apparatus of claim 1, further comprising a ceiling relative to said one or more sidewalls, and at least one of an anode or cathode in said ceiling and connected to said signal generator.

7. The apparatus of claim 6, further comprising a floor relative to said one or more sidewalls, and at least one of an anode or cathode in said ceiling and connected to said signal generator.

8. The apparatus of claim 7, wherein said conductive medium is hydrogel including a buffer solution providing conductivity.

9. The apparatus of claim 7, wherein said conductive medium is biological tissue.

10. A method of guiding nerve growth, the method including the steps of:
a. subjecting nerve tissue to a periodic AC signal at a desired frequency and desired amplitude chosen to facilitate nerve growth as compared to the growth of the nerve without the application of such a periodic AC signal, the periodic AC signal travelling from a current source to a ground; and
b. guiding growth of the nerve tissue by positioning the nerve tissue between the current source and ground to effect desired growth in a direction toward the ground.

11. The method of claim 10, wherein said step of subjecting nerve tissue to a periodic AC signal includes the application of an AC signal across multiple pairings of current sources and grounds.

12. The method of claim 11, wherein the application of AC signals across multiple pairings is controlled by creating desired pairings of current sources and grounds and controlling which pairing is active at any given time.

13. The method of claim 10, further comprising the steps of:
subjecting nerve tissue to a frustrating periodic AC signal at a desired frequency and desired amplitude chosen to frustrate nerve growth as compared to the growth of the nerve without the application of such a periodic AC signal, the periodic AC signal travelling from a current source to a ground.
